(19) United States
(12) Patent Application Publication
(10) Pub. No.: US 2025/0281711 A1
(43) Pub. Date: Sep. 11, 2025

(54) SUPPLEMENTAL OXYGEN GENERATOR

(71) Applicants: David Florman, Highland Heights, OH (US); Michael Florman, Pacific Palisades, CA (US)

(72) Inventors: David Florman, Highland Heights, OH (US); Michael Florman, Pacific Palisades, CA (US)

(21) Appl. No.: 19/071,738

(22) Filed: Mar. 5, 2025

Related U.S. Application Data
(60) Provisional application No. 63/561,329, filed on Mar. 5, 2024.

(51) Int. Cl. A61M 16/10; A61M 16/06; B01J 31/00; C12P 3/00

(52) U.S. Cl. CPC A61M 16/10; A61M 16/0672; B01J 31/003; C12P 3/00; A61M 2202/0007; A61M 2202/0208; A61M 2205/0216

(57) ABSTRACT

An oxygen generating system includes a vessel, an oxygen releasing ingredient, an oxygen activation ingredient, and an optional catalyst. The vessel has an outlet. When at least the oxygen releasing ingredient is mixed with the oxygen activation ingredient, oxygen is generated and is released through the outlet of the vessel.

### CROSS-REFERENCE TO RELATED APPLICATION

The present invention claims priority to U.S. Provisional Application No. 63/561,329, Mar. 5, 2024, the disclosure of which is incorporated herein by reference in its entirety.

### FIELD

The present invention relates to a supplemental oxygen generator. In particular, the present invention relates to a supplemental oxygen generator for human inhalation that is lightweight, portable, and easily used.

### BACKGROUND

Portable supplemental oxygen products are known. They typically come in prefilled bottles or canisters of oxygen that permit a user to breathe pure oxygen either by placing their mouth and/or nose adjacent the exit to the canister, or by using an attached oxygen mask or nasal cannulas. Typical canister size is 12 liters of concentrated oxygen providing users with 225 on-second inhalations. These oxygen products provide users with a number of one-second inhalations, such as 200 of 98%-99% pure breathing oxygen. Users use supplemental oxygen for all-natural respiratory support, emergency preparedness kits, poor air quality situations, traveling to areas of high altitude, and for enhancing athletic performance. While current products serve their purpose, the oxygen bottles currently available must be either thrown away or recycled. They cannot be reused. In addition, because they are prefilled with oxygen, they are under pressure, which may be undesirable in certain situations. Known supplemental oxygen canisters are typically constructed of aluminum and, as a result, are costly.

There are other portable oxygen generators on the market used by people with COPD, Asthma and other Respiratory ailments to provide supplemental oxygen. These devices generate oxygen using a concentrator that pulls in air from the surrounding environment. The air is compressed using a compressor and compressed air then passes through a sieve bed filter system. This system contains two chambers, each filled with a zeolite material. Zeolite is a porous material that has a strong affinity for nitrogen. When the compressed air passes through the sieve bed filters, the nitrogen molecules are attracted to the zeolite and are trapped. The oxygen molecules that remain then pass through the filters and are collected in a product tank. This oxygen is delivered to the patient through a nasal canula or mask. These types of devices typically produce between 5 and 10 liters of oxygen per minute, are large, and are costly. The present invention is directed toward a more environmentally friendly alternative that is also cost-effective, light weight, and portable.

### SUMMARY

An oxygen generating system includes a vessel, an oxygen releasing ingredient, an oxygen activation ingredient, and an optional catalyst. The vessel has an outlet. When at least the oxygen releasing ingredient is mixed with the oxygen activation ingredient, oxygen is generated and is released through the outlet of the vessel. The amount of oxygen that is generated is a function of the type of materials used in the system. The amount of time it takes to generate oxygen in the system is a function of the type of materials used in the system. The oxygen generating system may be portable and refillable. The oxygen generating system may be sealed and non-refillable.

A mask or nasal cannula may be attached to the outlet of the vessel to permit a user to inhale oxygen from the oxygen generating system.

### BRIEF DESCRIPTION OF THE DRAWINGS

FIG. 1 is a schematic of a first example inventive system described herein for generating supplemental oxygen;

FIG. 2 is a schematic of a second example inventive system described herein for generating supplemental oxygen;

FIG. 3 is an example refillable oxygen canister having a breathing mask attached directly thereto;

FIG. 4 is an example refillable oxygen canister having a breathing mask attached thereto via tubing;

FIG. 5 is an example refillable oxygen canister having nasal canula's attached thereto via tubing;

FIG. 6 is an example non-refillable oxygen canister in the form of a straw;

FIG. 7 is an example system having a refillable bottle along with two crushable ampules positioned in the bottle to assist in generating a reaction to generate oxygen; and

FIG. 8 is an example system having a refillable bottle along with a single crushable ampule positioned in the bottle and a lid for use in inhaling oxygen from the bottle.

### DETAILED DESCRIPTION

The present invention is directed to a device 10 and system 12 for generating oxygen via an oxygen releasing chemical reaction that occurs in a chamber, vessel, or reactor 14 that yields oxygen radicals and oxygen. The oxygen radicals and oxygen that are generated are breathable and clean. While previously known supplemental oxygen devices were closed and needed to be either thrown away or recycled after use, the present invention is directed toward a device 10 that can be reused multiple times to generate oxygen on the go by combining chemicals to generate oxygen for dispensing from a vessel, chamber or reactor 14. In addition, prior devices were prefilled with oxygen and, as a result, had contents under pressure. The present invention is directed toward a system 12 that generates oxygen when needed for immediate use.

For ease of description, the terms vessel, chamber or reactor 14 are used herein interchangeably to refer to an apparatus 14 designed to hold and dispense oxygen and to permit the generation of oxygen therein via a chemical reaction.

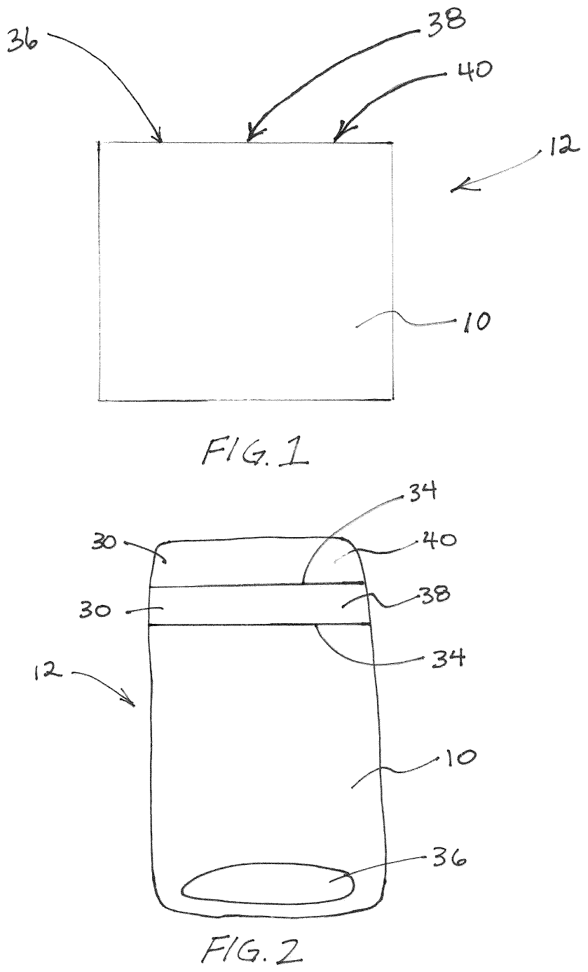

Referring to the figures, the apparatus 14 used in connection with the present invention can be any shape or size but is preferably small enough to easily carry around and light weight. FIG. 1 depicts the present system 12 as utilizing a vessel 14 having an input for receiving ingredients and an outlet 16 for dispensing oxygen. The vessel 14 has one or more openings 18 into which ingredients can be dispensed, and at least one outlet 18 for releasing oxygen. The vessel 14 may include a valve 10 coupled to the oxygen release port/exit 16 to control the release of oxygen from the vessel 14. The valve 20 may alternatively be positioned outside the vessel 14, such as on tubing 22 that emanates from the vessel 14. The valve 20 may be a check valve.

FIG. 2 depicts a container 14 having multiple compartments 24. The compartments 24 hold different ingredients that, when combined, generate oxygen. When the user is ready to utilize generated oxygen, the user can start the reaction process by permitting ingredients from different compartments 24 to interact with one another. This can occur by any known means, including by removing a separating member or piercing the various compartments to allow ingredients to interact.

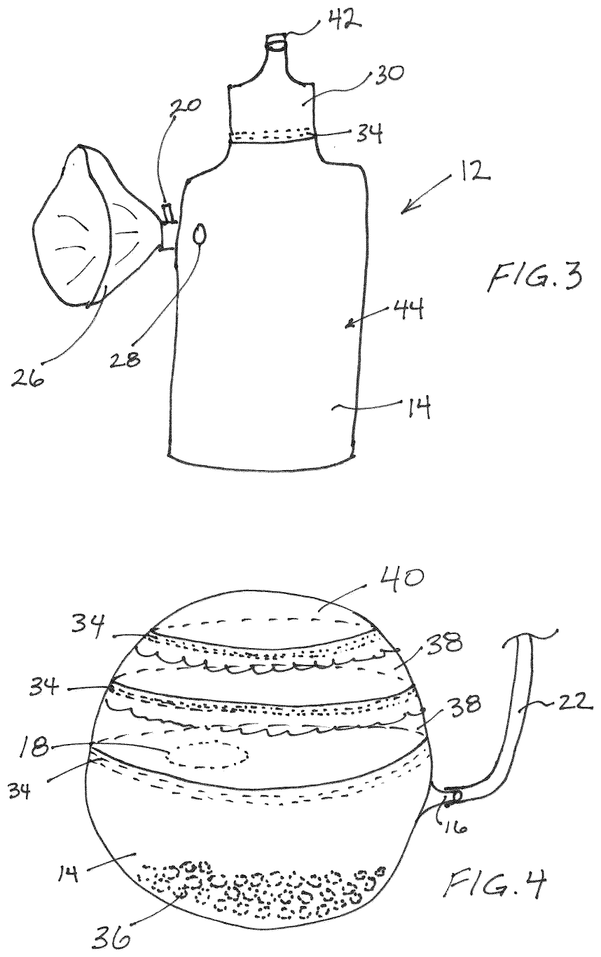

FIGS. 3-5 depict various vessels 14 that can be used with the present invention. FIG. 3 depicts a bottle-shaped container 10 having a seam that permits the top part of the container to be unscrewed to open the container 10 and to permit access to the compartment 24 of the container 10. This embodiment has a mask 26 attached to an outlet 16 of the container and a valve 20 may be associated with the mask 26 to permit oxygen to be released from vessel 14. The valve 20 may be associated with a wheel or a button, or with other means for opening the valve 20. For example, in one embodiment a user may press a button 28 to release oxygen from the vessel 14 and oxygen will be release as long as the user presses the button 28.

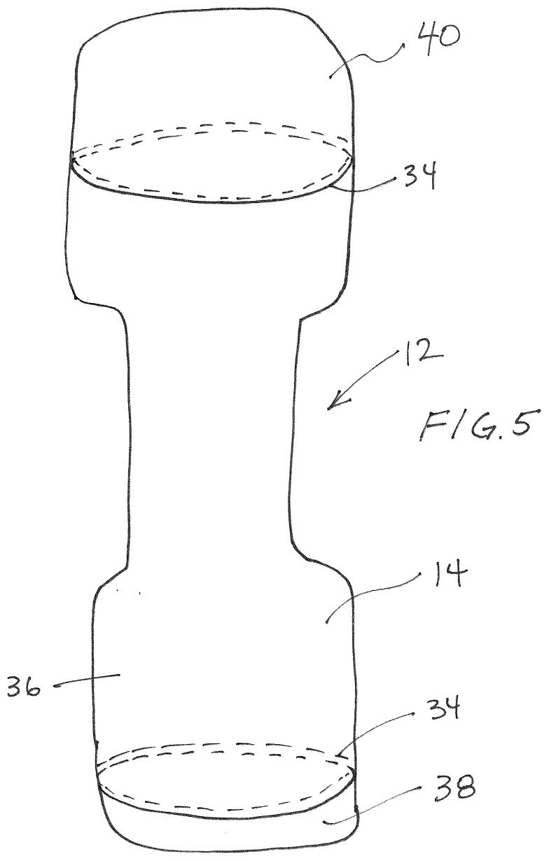

FIG. 4 depicts another example container 10 that is ball-shaped and could easily fit into the palm of a hand. This device 10 has a form factor that would permit a user to carry it during exercise, such as while jogging or walking. The ball-shaped vessel 14 has a screw or snap-on lid 30 that closes and opens the vessel 14 to permit ingredients to enter the vessel 14. The vessel 14 also has at least one exit where oxygen can be released from the vessel 14. The vessel 14 may have a port 18 into which a liquid or other ingredient can be poured that is separate from the screw or snap-on lid 30. This port 18 could be coupled to the lid 30 and have a smaller opening as shown in FIG. 4.

FIG. 5 is an alternative container 10 that is dumbbell shaped. It can be held in the hand and used during exercise, if desired. This vessel 14 also has a screw on top 30 and an optional port 18 for introducing ingredients into the container 10. The vessel 14 is shown with an exit port 16 to which tubing 22 may be attached and a nasal cannula 32, as shown in FIG. 8. The nasal cannula 32 could be worn continuously as oxygen is generated inside the vessel 14. The vessel 14 may have a shut-off valve 20 to turn on and off the oxygen output. As is evident, the vessel 14 can have any desired shape.

While not shown, the vessel 14 may be an open container. Although oxygen will not be stored therein, oxygen can be generated in a burst to permit immediate use by a user.

The present system utilizes an oxygen releasing chemical reaction that occurs in the vessel 14 that yields oxygen radicals and oxygen. The oxygen that is generated is clean and breathable. The reaction may occur in a single chamber container or a multiple chamber container.

The vessel 14 may be made from a hard or soft materials that can include one or more of plastic, metal, natural fibers, paper, cardboard, or any other material that can be fabricated into a chamber to hold chemical ingredients and/or water.

The vessel 14 may be sealed with no access to open the vessel 14 after fabrication or could be resealable by the user, allowing the vessel 14 to be filled and emptied and refilled. As discussed above, the vessel 14 can have a shut off valve 20 that allows the user to open and close the vessel 14. The user opens the valve 20 to allow the release of oxygen from the vessel 14 and closes the valve 20 to trap the oxygen in the vessel 14 for later use.

The vessel 14 may have a lid or cap 30 that is attached to the remainder of the vessel 14 with standard fittings, such as screw threads, snap lock, or luer lock 34 fittings. Alternatively, custom fittings can be used for closure. Furthermore, the vessel 14 may have custom fittings attached to the exterior thereof to allow accessories to be attached to the vessel 14. The vessel 14 may use any one of the standard fittings as attachment mechanisms for attaching things to the vessel 14.

The vessel 14 may be prefilled with an oxygen releasing agent 36, if desired. Alternatively, the vessel 14 may be empty and then filled with an oxygen releasing agent 36. There are numerous types of oxygen releasing agents 36. The present invention may utilize one or more of the following: Hydrogen peroxide (H₂O₂), Potassium chlorate (KCₗO₃), Sodium peroxide (Na₂O₂), Potassium superoxide (KO₂), Barium peroxide (BaO₂), Sodium perborate, Sodium carbonate peroxyhydrate, Magnesium peroxide, Calcium peroxide, Potassium perborate, Ammonium perborate, Sodium perphosphate, Sodium persulfate, Carbamide peroxide, sodium percarbonate, benzoyl peroxide, organic peroxides, peracetic acid, calcium peroxide, magnesium peroxide, potassium peroxide, barium peroxide, zinc peroxide, strontium peroxide, acetyl peroxide, and acetone peroxide. Other ingredients may alternatively be utilized, as long as they are capable of generating oxygen when combined with one or more other ingredients.

The oxygen releasing agents 36 can be preinstalled in the vessel 14 or can be added to the vessel 14 when oxygen generation is desired. As discussed above, the oxygen releasing agents 36 may comprise one or more agents that are mixed together.

To activate the generation of oxygen, these agents need to be activated by adding water or other activating chemicals 38 that react with the oxygen releasing agent 36 to generate oxygen. The activation chemical 38 is added to the releasing agent 36 to begin the chemical process. A catalyst 40 can also be added to the activation chemical 38 in the vessel 14 to accelerate oxygen generation. For example, a catalyst 40, such as potassium iodide, can be used to accelerate the breakdown of sodium percarbonate and water to generate oxygen from the sodium perchlorate. Catalysts 40 may include one or more of the following:
1. enzymes, including, but not limited to, catalase and peroxidase derived from natural sources of fruits and vegetables and animal liver;
2. transition metal ions;
3. potassium and/or sodium iodide;
4. silver nanoparticles; and
5. molybdenum compounds.

Depending on the quantity of oxygen releasing agent utilized and the type of ingredients utilized as well as sequence of adding the ingredients, a finite amount of oxygen will be generated over a period time, dependent on the following formula:

$$Quantity of Oxygen Releasing Agent 36 + Quantity of Activating Agent 38 = Total Maximum Oxygen Released or Generated$$

Increasing or decreasing the amounts of the Oxygen Releasing Agent 36 and Activating Agent 38 will increase or decrease the amount of oxygen generated. Use of a catalyst 40 can speed up the process of oxygen generation.

The amount of catalyst 40 and type of catalyst 40 used will determine the rate of total exhaustion of the reaction. By increasing or decreasing the amount of catalyst 40, the total reaction time will increase or decrease proportionately. Depending on the desired speed of the oxygen generating reaction, the amount of catalyst 40 can be increased or decreased to cause the oxygen generating ingredient 36 along with the Activating agent 38 to release all of its oxygen in a specific amount of time, as desired by the manufacturer or end user. Prefilled bottles can be utilized to identify a certain amount of oxygen to be produced in a specified amount of time. For example, if someone is exercising, such as a 30-minute run, the user can titrate the catalyst 40 to have the oxygen delivered and depleted in that time frame. If a time period of 3 hours is desired, such as for a person who needs oxygen when they are performing a task (like shopping at the mall or going out to dinner), the bottles may be set to operate for 3 hours and then the reaction will end/expire.

As discussed above, the vessel 14 may be a single use vessel 14 or a reusable vessel 14. The combination of the vessel 14, oxygen releasing agent 36 and activating agent 38/catalyst 40 can be sold or marketed based upon total oxygen available or total time period of oxygen generation.

Numerous types of fittings can be attached to the vessel 14. For example, a mask 26 that can be positioned to cover the mouth and/or nose can be used. Nasal cannulas 32 can be used. A nasal inhaler 42 can be used such that the user inserts the inhaler 42 into the nose 44 and snorts the oxygen being fabricated. Alternatively, instead of a mask 26, a partial enclosure can be used to apply the vessel 14 against a user's skin to enhance wound healing. Oxygen helps improve blood flow to the wound, which delivers more oxygen and nutrients to the healing tissue. The fittings can be worn during sleep or while awake, during exercise and/or rest or work.

A flavor or scent can be added to the vessel 14, which can include artificial scents or flavors, or natural scents and flavors. Examples of scents or flavors that may be utilized include: Essential oils, Lavender oil, Peppermint oil, Tea tree oil, Lemon oil, Eucalyptus oil, Frankincense oil, Rosemary oil, Chamomile oil, Lemongrass oil, Ylang ylang oil, Bergamot oil, Geranium oil, Cedarwood oil, Sandalwood oil, Clary sage oil, Lime oil, Grapefruit oil, Neroli oil, Jasmine oil, Natural flavors derived from natural sources, Artificial flavors, Fruit flavors, Citrus flavors, Herbal flavors, Sweet flavors, Savory flavors, Bitter flavors, Spicy flavors, and Floral flavors.

Lavender oil is known for its calming and relaxing properties. It is often used in aromatherapy to promote relaxation and alleviate stress and anxiety. Peppermint oil has a refreshing and invigorating scent. It is commonly used to soothe headaches, relieve nausea, and improve focus and concentration. Tea tree oil has antiseptic and antimicrobial properties, making it useful for treating acne, skin infections, and minor cuts and wounds. It is also used in natural cleaning products. Lemon oil has a fresh and uplifting scent. It is used in aromatherapy to boost mood and energy levels. Lemon oil is also used as a natural cleaner and flavoring agent in food and beverages. Eucalyptus oil has a cool and refreshing aroma. It is commonly used to relieve congestion and promote clear breathing. Eucalyptus oil is also used in topical applications to soothe sore muscles and joints.

Frankincense oil has a warm and woody aroma. It is often used in aromatherapy to promote relaxation and spiritual well-being. Frankincense oil is also used in skincare products for its rejuvenating properties. Rosemary oil has a fresh and herbaceous scent. It is used to improve memory, concentration, and mental clarity. Rosemary oil is also used in hair care products to promote scalp health and stimulate hair growth. Chamomile oil has a sweet and floral aroma. It is known for its calming and soothing properties, making it useful for promoting relaxation and restful sleep. Lemongrass oil has a citrusy and uplifting scent. It is used to relieve stress, reduce inflammation, and repel insects. Lemongrass oil is also used in culinary applications for its flavor and aroma. Ylang ylang oil has a sweet and floral fragrance. It is used in aromatherapy to reduce anxiety, promote relaxation, and enhance mood. Ylang ylang oil is also used in skincare products and perfumes for its exotic aroma.

Bergamot oil has a citrusy and floral aroma. It is used to uplift mood, reduce stress and anxiety, and promote relaxation. Bergamot oil is also used in skincare products for its cleansing and purifying properties. Geranium oil has a sweet and floral scent with hints of rose. It is used to balance emotions, relieve stress, and promote feelings of well-being. Geranium oil is also used in skincare products for its astringent and moisturizing properties. Cedarwood oil has a warm and woody aroma. It is used to promote relaxation, reduce stress, and support sleep. Cedarwood oil is also used in skincare products to soothe and moisturize the skin. Patchouli oil has a rich, earthy, and musky aroma. It is used to promote relaxation, relieve stress, and enhance mood. Patchouli oil is also used in skincare products for its anti-inflammatory and antiseptic properties.

Sandalwood oil has a sweet, woody, and exotic scent. It is used to promote relaxation, reduce anxiety, and enhance meditation. Sandalwood oil is also used in skincare products for its soothing and hydrating properties. Clary sage oil has a sweet and herbal aroma. It is used to balance hormones, relieve menstrual discomfort, and reduce stress and anxiety. Clary sage oil is also used in skincare products for its antibacterial and anti-inflammatory properties. Lime oil has a fresh, citrusy, and uplifting scent. It is used to energize the mind, uplift mood, and promote mental clarity. Lime oil is also used in skincare products for its astringent and cleansing properties. Grapefruit oil has a sweet, citrusy, and invigorating aroma. It is used to boost energy, uplift mood, and reduce stress and anxiety. Grapefruit oil is also used in skincare products for its cleansing and toning properties. Neroli oil has a sweet, floral, and exotic scent. It is used to reduce stress, promote relaxation, and improve sleep quality. Neroli oil is also used in skincare products for its rejuvenating and regenerative properties. Jasmine oil has a rich, floral, and intoxicating scent. It is used to uplift mood, reduce anxiety, and enhance romantic feelings. Jasmine oil is also used in skincare products for its moisturizing and soothing properties.

Natural flavors are derived from natural sources such as fruits, vegetables, herbs, spices, and other plant and animal sources. These flavors are extracted through various methods such as cold pressing, distillation, or solvent extraction. Examples include strawberry, vanilla, mint, and citrus flavors. Artificial flavors are synthesized in laboratories using synthetic chemicals to mimic the taste of natural flavors. These flavors are often used to enhance or modify the taste of food and beverage products. Examples include artificial strawberry, artificial vanilla, and artificial citrus fruit flavors.

Fruit Flavors are derived from various fruits and are used to impart the taste of specific fruits to food and beverage products. Examples include apple, banana, pineapple, and berry flavors. Citrus flavors are derived from citrus fruits such as oranges, lemons, limes, and grapefruits. These flavors are characterized by their tangy and refreshing taste and are commonly used in beverages, candies, and desserts. Herbal flavors are derived from herbs and spices such as mint, basil, cinnamon, and ginger. These flavors add depth and complexity to food and beverage products and are often used in savory dishes, teas, and herbal supplements.

Sweet Flavors are characterized by their sugary taste and are often used to enhance the sweetness of food and beverage products. Examples include caramel, chocolate, honey, and maple flavors. Savory flavors are characterized by their umami taste and are often used to enhance the savory or meaty notes of food products. Examples include beef, chicken, mushroom, and cheese flavors. Bitter flavors are characterized by their bitter taste and are often used to balance the sweetness of food and beverage products. Examples include coffee, dark chocolate, and bitter herbs such as arugula and radicchio. Spicy flavors are characterized by their heat and pungency and are often derived from spices such as chili peppers, black pepper, and paprika. These flavors add warmth and intensity to food and beverage products. Floral flavors are derived from flowers such as roses, lavender, and jasmine. These flavors add a delicate and aromatic quality to food and beverage products and are often used in teas, confections, and perfumes.

The percentage of flavor or scent can range from about 0.01% to about 30% depending on the amount of aroma intended to be emitted. In addition, medicaments such as menthol, or vitamins, and the like can be added to the vessel 14.

There are numerous other types of vessels 14 that can utilize the technology described herein. For example, the water chamber in a CPAP machine can be used as the reactor for the chemical reaction generating oxygen by adding the oxygen releasing agent 36 to the water 38 in the CPAP machine. The vessel 14 can be a simple bottle 44 that the end user opens and the oxygen releasing agent 36 sits loose in the bottle 44, and the end user uses tap water or bottled water 38 to activate the oxygen agent by pouring the water into the bottle 44 to a specific fill line, eliminating the need to ship the product with water.

The vessel 14 can contain one chamber 24 with multiple sachets, ampules, or unit dose packs 46 inside the chamber 24 that keep the oxygen releasing agent 36 separated from the activator 38. A second unit dose pack 46 can be used for a catalyst 40. When the end user wishes to activate the oxygen releasing agent 36 with the activator 38 and/or catalyst 40, the user either opens the vessel 14 and removes the sachets 46 from the vessel 14 and pours the contents back into the reactor to mix the contents or crushes the sachets 46 inside the reactor and mixes the contents. In one example, the sachets 46 can be thin glass ampules that can be broken inside the reactor by pressing on the outside of the vessel 14. That way, it is not necessary for the user to open the bottle 44 and handle the contents.

In one embodiment, the vessel 14 has multiple chambers 24. These chambers 24 can be defined by different openable containers or by sealed containers, such as breakable ampules 46. An outer chamber can hold the oxygen releasing ingredient 36 while two inner chambers can include an oxygen activating ingredient 38 (such as water) and a catalyst 40. The inner chambers can be coupled to one another so that when both are opened or broken open, they will mix with the oxygen releasing ingredient 36. Alternatively, the inner chambers can be separated from one another so that only the oxygen activating ingredient 38 is permitted to mix with the oxygen releasing ingredient 36. In this way, one container 10 can provide different time periods for oxygen generation depending on which container is opened and added to the outer chamber.

The chambers 24 could be positioned adjacent one another in a single vessel 14, such as a larger vessel 14 where the reaction occurs (reactor), and several attached additional vessels. An example of this is shown in FIG. 5. In this embodiment, the additional vessels can be opened and the contents added to the larger vessel 14.

Alternatively, the vessel 14 may have multiple chambers 24 and the chambers 24 can be separated by one or more dividers that are either removable or fixed in position. The intent of the dividers is to keep the contents of the chambers 24 separated until the user wishes to combine the ingredients together by forcing one of the ingredients through the divider. The divider could be a membrane that is torn by pressure, for example.

If desired, an additional ingredient can be utilized that, when mixed with the oxygen activating ingredient 38 and/or oxygen releasing ingredient 36, generates an exothermic reaction. This produces heat, which can be utilized to speed up the oxygen releasing reaction. A similar type of process is presently used by commercial airlines to produce oxygen where a peroxide or sodium chlorate (NaClO₃) is heated by an explosion to release the oxygen from the peroxide compound. When heated Sodium Chlorate decomposes and releases oxygen. The oxygen generator used by airlines is located above each row of seats and when cabin altitude reaches a predetermined height, or the system is activated by the flight crew, overhead panels open and oxygen masks drop down. Pulling down the mask removes the retaining pins and triggers the production of oxygen. The reaction nis exothermic and the exterior temperature of the generator reaches 500° F. This generator will produce oxygen for 15 minutes. This system usually uses mainly Sodium Chlorate (NaClO₃) mixed with less than 5% Barium Peroxide (BaO₂) and less than 1% Potassium Perchlorate (KClO₄).

The formulation of chemicals in the reactor can contain a sweetener that is either a natural sweeteners or an artificial sweetener. The artificial sweetener may be one or more of the following: aspartame, sucralose, saccharin, acesulfame potassium, neotame, or other artificial sweeteners. The natural sweetener may include one or more of: sucrose, honey, maple syrup, agave nectar, stevia, or other natural sweeteners. The sweetener may be derived from corn, such as high fructose corn syrup, corn syrup, corn sweetener, corn sugar (dextrose), glucose syrup, liquid glucose, dextrose anhydrous, dextrose monohydrate, and similar compounds. The percentage of sweetener can range from about 0.01% to about 30%. Other sweeteners may alternatively be utilized, the invention not being limited to those described herein.

The formulation may include aa artificial or natural coloring agent that changes color as the oxygen reaction is occurring, allowing the user to know when the oxygen reaction is exhausted. For example, the color could change from a color (e.g., pink, yellow, purple, blue) to clear, with clear indicating the exhaustion of the oxygen.

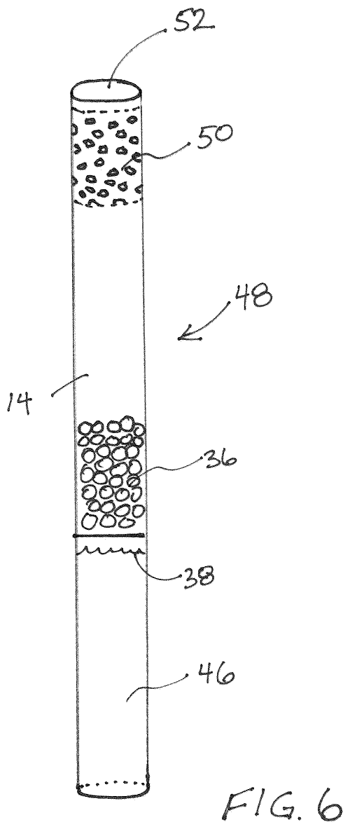

Referring to FIG. 6, the vessel 14 may be made of plastic or other flexible materials that is in the shape of a sealed straw 48 on one end and a glass ampule 46 or multiple glass ampules 46 inside the flexible, single ended, sealed straw 48. A wick or sponge 50 could be positioned at the open end 52 of the straw 48. When the glass ampule 46 is crushed inside the straw 48, the liquid 38 inside the ampule 46 mixes with the oxygen releasing agent 36 and releases oxygen through the end 52 of the straw 48 having the wick or sponge 50. The wick or sponge 50 deters the liquid mixture from pouring out of the open end 52 of the straw 48, while also filtering the mixture by keeping the oxygen releasing agent 36 and its components from reaching the end users nose as the end user sniffs the oxygen.

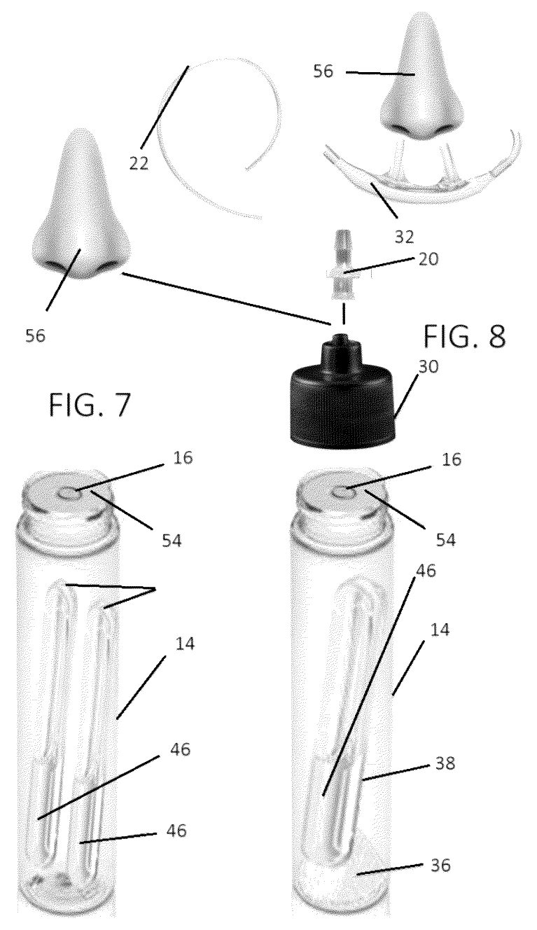

FIG. 7 depicts an alternative embodiment where a vessel 14 has two breakable ampules 46 positioned inside the vessel 14, along with a vented cap liner 54 onto which a lid or cap 30 can be seated. The vessel 14 is a flexible plastic bottle 44 having two crushable glass ampules 46. One of the ampules 46 contains a liquid catalyst 40 component or another ingredient, such as an aroma component. The other ampule 46 may contain a peroxide liquid component 38, for example. An oxygen releasing powder material 36 may be positioned inside the flexible plastic bottle 44 so that when the user crushes the peroxide liquid component 38, it mixes with the oxygen releasing powder material 36 to generate oxygen.

FIG. 8 is an exploded view of the oxygen generating system 12 showing a flexible plastic bottle 44 containing a crushable glass ampule 46 containing a water component 38 that also includes a catalyst 40. The water 38 and catalyst 40 may be combined in a single ampule 44, or the ampule 44 may have two or more separate chambers 24. The bottle includes a vented cap liner 54 having a vent 18, and a bottle cap 30 having a leur fitting 34 or connector is attached to the upper end of the bottle 30 around the vent 18. The vent 18 communicates with an opening 16 in the bottle cap 30. The bottle cap 30 has a connector having an outlet 16 with a valve 20 that can be opened and closed. The connector outlet 16 can be inserted directly into a user's mouth or nose 56. The connector outlet 16 can be coupled to a leur fitting that permits a hose or tubing 22 to be connected to the leur fitting, and a cannula 32 or mask 26 can be attached to the hose 22. In use, the user will crush the ampule 46 inside the flexible bottle 44 by applying pressure to an exterior surface of the bottle 44. This will then cause the oxygen activating ingredient 38 (and catalyst 40 if included) to mix with the oxygen releasing powder 36 positioned inside the bottle 44 to generate oxygen. Oxygen will be generated over time as the oxygen releasing powder 36 interacts with the oxygen activating material 38. Any generated oxygen may be stored in the bottle under pressure when the bottle cap valve 20 is in a closed position. When the bottle cap valve 20 is opened, oxygen may be released.

The bottle/vessel 14 may be transparent and flexible and the ampules/breakable containers 46 may be labeled so that the user can determine which container to break. For example, the oxygen releasing ingredient 36 may be positioned in the vessel 14 and multiple ampules 46 of oxygen activating ingredients 38 may be positioned inside the container and/or multiple containers of catalyst 40 may be positioned inside the container 14. The user may break one of the oxygen activating containers 38 by pressing on the flexible container. The user may, at the same time, break one of the catalysts 40 if desired to speed up the oxygen generation process. After or as the oxygen activating ingredient 38 is spent, the user may break the second oxygen activating ingredient 38 and the catalyst 40 (if desired) to continue the oxygen generation process. As is evident, an amount of oxygen releasing ingredient 36 must be sufficient for use with two or more breakable containers so that the oxygen releasing ingredient 36 is not already spent when the second breakable container 46 is broken.

A toilet can be used as the reactor for the chemical reaction generating oxygen by adding the oxygen releasing agent 36 to the toilet before or after using the toilet. A sink drain trap can be used as the reactor by placing the oxygen releasing agent 36 into the sink, running some water into the drain and allowing the agents to sit in the trap. A user's mouth could be used as the reactor by placing the oxygen releasing agent 36 into the mouth, mixing the agent with saliva in the mouth, and swishing or brushing one's teeth with a toothbrush, then expectorating.

The percentage of oxygen releasing agent 36 can range from about 100% down to about 1% depending on the amount of oxygen desired to be released.

Filler agents can be added and mixed with the oxygen releasing agent 36 in the range of about 99% down to about 1%. Filler agents can be inert chemicals such as silica, or minerals such as sodium carbonate, sodium bicarbonate, aluminum hydroxide, magnesium hydroxide, calcium carbonate, sodium silicates, talc, kaolin-clay, barite, diatomaceous earth, bentonite, gypsum, nylon-12, mica, boron nitride, starch (corn, tapioca, rice, potato, arrowroot, wheat), etc.

Where hydrogen peroxide is utilized as the oxygen releasing agent 36, objects can be inserted into the reactor 14 and cleaned as oxygen radicals are being released from the hydrogen peroxide molecule.

A cleaner or surfactant can be added to the reactor 14. The surfactant can be chosen from any surfactants used in cosmetics and pharmaceuticals manufacturing from the following categories: Ionic, nonionic, cationic, bio, and amphoteric. The percentage of surfactant can range from about 0.01% to about 5%.

The vessel 14 can be worn with clothing and be attached by a clip or pin or other method of attachment. The vessel 14 can be worn on a lanyard or necklace. The vessel 14 can be attached to the person's belt. The vessel 14 can be placed in a bag, such as a handbag, backpack or fanny pack. The vessel 14 can be placed in a bicycle storage container or in a water bottle holder type holder.

The reaction occurring in the vessel 14 can be accelerated by using warm or hot water. The reaction occurring in the vessel 14 can be accelerated by shaking the reactor to mix the solution. Shaking is not traditionally used when releasing oxygen releasing agents 36 because most products presently available that release oxygen do not recommend shaking to mix. In the present case, shaking can aid in mixing the ingredients to make the reaction more efficient and effective. Shaking can occur naturally as users carry the vessel 14 while they are exercising, leveraging natural movements associated with exercise. Positioning the vessel 14 next to one's body also may increase the reaction by utilizing body heat to accelerate the oxygen release.

In one embodiment, the reactor can be plumbed into a CPAP machine between the CPAP machine and the CPAP mask using a “T” or similar adapter into the CPAP hose.

Other oxygen releasing chemicals 36 may be utilized with the invention, including but not limited to potassium peroxymonosulfate or sodium monopersulfate.

The vessel 14 or reactor utilized herein can be designed to hold the oxygen being generated under pressure so that the chamber 24 can be filled very quickly by adding water and closing the lid of the chamber 24. A valved system or regulator 20 can be incorporated into the system to control the amount of oxygen being released.

According to the invention, an oxygen generating system includes a vessel 14, an oxygen releasing ingredient 36, an oxygen activation ingredient 38, and an optional catalyst 40. The vessel 14 has an outlet 16. When at least the oxygen releasing ingredient 36 is mixed with the oxygen activation ingredient 38, oxygen is generated and is released through the outlet 16 of the vessel 14.

The oxygen generating system yields oxygen radicals and oxygen and the oxygen is non-toxic and breathable. The vessel 14 is a single chamber or the vessel 14 has multiple chambers. At least one of the vessel 14 is refillable, the vessel 14 is not refillable, and the vessel 14 is portable. The vessel 14 may be fabricated out of hard or soft materials that comprise a material that is one or more of plastic, metal, natural fibers, paper, cardboard, or glass and is capable of holding at least one chemical or water. The outlet 16 of the vessel 14 may include a valve 20 for opening and closing the outlet 16.

The oxygen releasing ingredient 36 may include one or more of Hydrogen peroxide (H₂O₂), Potassium chlorate (KClO₃), Sodium peroxide (Na₂O₂), Potassium superoxide (KO₂), Barium peroxide (BaO₂), Sodium perborate, Sodium carbonate peroxyhydrate, Magnesium peroxide, Calcium peroxide, Potassium perborate, Ammonium perborate, Sodium perphosphate, Sodium persulfate, Carbamide peroxide, sodium percarbonate, benzoyl peroxide, organic peroxides, peracetic acid, calcium peroxide, magnesium peroxide, potassium peroxide, barium peroxide, zinc peroxide, strontium peroxide, acetyl peroxide, acetone peroxide, peroxymonosulfate and sodium monopersulfate.

The oxygen activation ingredient 38 is water or another liquid ingredient. The water or liquid ingredient reacts with the oxygen releasing ingredient 36 to yield oxygen.

The catalyst 40 may be included and accelerates oxygen generation by accelerating the breakdown of the oxygen releasing ingredient 36. The catalyst 40 can include one or more of enzymes, transition metal ions, potassium iodide, sodium iodide, silver nanoparticles, and molybdenum compounds. The enzymes include one or more of catalase and peroxidase derived from natural sources of fruits and vegetables and animal liver.

An amount of oxygen created by the system is dependent on the quantity and type of oxygen releasing ingredient 36 and oxygen activation ingredient 38. The amount of oxygen may be generated over a time period and the time period may be a function of the amount of oxygen releasing ingredient 36, the amount of oxygen activating ingredient 38, and whether a catalyst 40 is used. Inclusion of the catalyst 40 will decrease the amount of time needed to generate oxygen.

The system may also include tubing and a nasal cannula attached to the outlet 16, or a mask 26 attached to the outlet 16, or a nasal inhaler fitting that permits a user to insert the inhaler into a user's nose or mouth to snort oxygen being generated. The system may also include a first additional ingredient in the form of a scent, flavor, or essential oil positioned in the vessel 14, including one or more of lavender oil, peppermint oil, tea tree oil, lemon oil, eucalyptus oil, frankincense oil, rosemary oil, chamomile oil, lemongrass oil, ylang ylang oil, bergamot oil, geranium oil, cedarwood oil, patchouli oil, sandalwood oil, clary sage oil, lime oil, grapefruit oil, neroli oil, jasmine oil, natural flavors, artificial flavors, fruit flavors, citrus flavors, herbal flavors, sweet flavors, savory flavors, bitter flavors, spicy flavors, and floral favors. The system may also include a second additional ingredient comprising one or more of menthol, vitamins, or medicines. The system may also include a third additional ingredient comprising a sweetener comprising one or more of an artificial sweetener including aspartame, sucralose, saccharin, acesulfame potassium, and neotame, a natural sweetener including sucrose, honey, maple syrup, agave nectar, and stevia, a sweetener derived from corn including high fructose corn syrup, corn syrup, corn sweetener, dextrose, glucose syrup, liquid glucose, dextrose anhydrous, and dextrose monohydrate, with the third additional ingredient being in the range of 0.01% to 30% of the oxygen generated. The system may also include a fourth additional ingredient comprising a color changing ingredient that changes a color of the oxygen generated from colored to clear.

The vessel 14 may be a chamber in a CPAP machine. The vessel 14 may include multiple compartments that are separated by breakable members. The vessel 14 may include multiple compartments that comprise individual vessels 14 that are coupled together by a fastening means.

The vessel 14 may be a cylindrical, flexible member in the shape of a straw having an interior, an open end and a closed end. A breakable, sealed ampule may be positioned at the closed end housing the oxygen activating material and in communication with the interior of the cylindrical member. The ampule houses the oxygen releasing material. When the ampule is broken inside the cylindrical, flexible member, the oxygen activating material mixes with the oxygen releasing ingredient 36 to generate oxygen, which is released through the open end of the member.

Other chemical compositions may be utilized with the invention described herein. The ingredients described herein are for illustrative purposes and are non-exhaustive. The various ingredients described herein may be optimized to provide a maximum amount of oxygen based upon the percentage of ingredients utilized. The percentage of each ingredient may be equal or different.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the aspects described are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim. The term “consisting essentially,” if used herein, means the specified materials or steps and those that do not materially affect the basic and novel characteristics of the material or method. The articles “a,” “an,” and “the,” should be interpreted to mean “one or more” unless the context indicates the contrary.

What is claimed is:

1. An oxygen generating system comprising:
a vessel having an outlet;
an oxygen releasing ingredient;
an oxygen activation ingredient; and
an optional catalyst,
wherein when at least the oxygen releasing ingredient is mixed with the oxygen activation ingredient, oxygen is generated and is released from the vessel through the outlet.

2. The system of claim 1, wherein the oxygen generating system yields oxygen radicals and oxygen and the oxygen is non-toxic and breathable.

3. The system of claim 1, wherein the vessel is a single chamber or the vessel has multiple chambers.

4. The system of claim 1, wherein at least one of the vessel is refillable, the vessel is not refillable, and the vessel is portable.

5. The system of claim 1, wherein the vessel is fabricated out of hard or soft materials that comprise a material that is one or more of plastic, metal, natural fibers, paper, cardboard, or glass and is capable of holding at least one liquid chemical or water.

6. The system of claim 1, wherein the outlet of the vessel includes a valve for opening and closing the outlet.

7. The system of claim 1, wherein the oxygen releasing ingredient comprises one or more of Hydrogen peroxide (H2O2), Potassium chlorate (KClO3), Sodium peroxide (Na2O2), Potassium superoxide (KO2), Barium peroxide (BaO2), Sodium perborate, Sodium carbonate peroxyhydrate, Magnesium peroxide, Calcium peroxide, Potassium perborate, Ammonium perborate, Sodium perphosphate, Sodium persulfate, Carbamide peroxide, sodium percarbonate, benzoyl peroxide, organic peroxides, peracetic acid, calcium peroxide, magnesium peroxide, potassium peroxide, barium peroxide, zinc peroxide, strontium peroxide, acetyl peroxide, acetone peroxide, peroxymonosulfate and sodium monopersulfate.

8. The system of claim 1, wherein the oxygen activation ingredient is a liquid chemical or water and the liquid chemical or water reacts with the oxygen releasing ingredient to yield oxygen.

9. The system of claim 1, wherein the catalyst is included and the catalyst accelerates oxygen generation by accelerating the breakdown of the oxygen releasing ingredient.

10. The system of claim 9, wherein the catalyst comprises one or more of enzymes, transition metal ions, potassium iodide, sodium iodide, silver nanoparticles, and molybdenum compounds.

11. The system of claim 10, wherein the enzymes include one or more of catalase and peroxidase derived from natural sources of fruits and vegetables and animal liver.

12. The system of claim 1, wherein an amount of oxygen created by the system is dependent on the quantity and type of oxygen releasing ingredient and oxygen activation ingredient.

13. The system of claim 12, wherein the amount of oxygen is generated over a time period and the time period is a function of the amount of oxygen releasing ingredient, the amount of oxygen activating ingredient, and whether a catalyst is used.

14. The system of claim 1, wherein the inclusion of the catalyst will decrease the amount of time needed to generate oxygen.

15. The system of claim 1, further comprising tubing and a nasal cannula attached to the outlet, or a mask attached to the outlet, or a nasal inhaler fitting that permits a user to insert the inhaler into a user's nose to snort oxygen being generated.

16. The system of claim 1, further comprising a first additional ingredient in the form of a scent, flavor, or essential oil positioned in the vessel, including one or more of lavender oil, peppermint oil, tea tree oil, lemon oil, eucalyptus oil, frankincense oil, rosemary oil, chamomile oil, lemongrass oil, ylang ylang oil, bergamot oil, geranium oil, cedarwood oil, patchouli oil, sandalwood oil, clary sage oil, lime oil, grapefruit oil, neroli oil, jasmine oil, natural flavors, artificial flavors, fruit flavors, citrus flavors, herbal flavors, sweet flavors, savory flavors, bitter flavors, spicy flavors, and floral favors; and/or
a second additional ingredient comprising one or more of menthol, vitamins, or medicines; and/or
a third additional ingredient comprising a sweetener comprising one or more of an artificial sweetener including aspartame, sucralose, saccharin, acesulfame potassium, and neotame, a natural sweetener including sucrose, honey, maple syrup, agave nectar, and stevia, a sweetener derived from corn including high fructose corn syrup, corn syrup, corn sweetener, dextrose, glucose syrup, liquid glucose, dextrose anhydrous, and dextrose monohydrate, with the third additional ingredient being in the range of 0.01% to 30% of the oxygen generated; and/or
a fourth additional ingredient comprising a color changing ingredient that changes a color of the oxygen generated from colored to clear.

17. The system of claim 1, wherein the vessel is a chamber in a CPAP machine.

18. The system of claim 1, wherein the vessel includes multiple compartments that are separated by breakable members, or the multiple compartments comprise individual vessels that are coupled together by a fastening means.

19. The system of claim 1, wherein the vessel is a cylindrical, flexible member in the shape of a straw having an interior, an open end and a closed end, with a breakable, sealed ampule positioned at the closed end housing the oxygen activating material and in communication with the interior of the cylindrical member housing the oxygen releasing material, and when the ampule is broken inside the cylindrical, flexible member, the oxygen activating material mixes with the oxygen releasing ingredient to generate oxygen, which is released through the open end of the member.
